(19) United States
(12) Patent Application Publication
(10) Pub. No.: US 2019/0232232 A1
(43) Pub. Date: Aug. 1, 2019

(54) BIOCOMPATIBLE AND HEMOCOMPATIBLE MATERIAL AND FILTER

(71) Applicant: QIDNI LABS, INC., San Francisco, CA (US)

(72) Inventors: Andrew MENDONCA, Waterloo (CA); Raman M. SUD, Waterloo (CA); Morteza AHMADI, Waterloo (CA); Taimoor KHAN, Waterloo (CA)

(73) Assignee: QIDNI LABS, INC., San Francisco, CA (US)

(21) Appl. No.: 16/312,089

(22) PCT Filed: Jul. 14, 2017
(86) PCT No.: PCT/US2017/042173

§ 371 Date: Dec. 20, 2018

Related U.S. Application Data
(60) Provisional application No. 62/362,556, filed on Jul. 14, 2016.
(60) Provisional application No. 62/362,560, filed on Jul. 14, 2016.

(51) Int. Cl. B01D 69/12; B01D 61/14; B01D 61/24; B01D 71/02; B01D 67/00; B01D 69/06; B01D 69/04; C23C 16/04; C23C 16/26; C23C 16/56; A61M 1/34; A61L 31/06; A61M 1/36; A61M 1/16

(52) U.S. Cl. CPC B01D 69/12; A61M 2202/0498; B01D 61/243; B01D 71/021; B01D 71/024; B01D 67/0067; B01D 69/06; B01D 69/04; C23C 16/045; C23C 16/26; C23C 16/56; A61M 1/34; A61L 31/06; A61M 1/3661; A61M 1/1678; A61M 2205/04; A61M 2207/10; A61M 2210/12; B01D 61/145

(57) ABSTRACT

A biocompatible and hemocompatible material and filter which is suitable for blood filtration applications. Biocompatibility and hemocompatibility is achieved through a modification of an existing ceramic substrate, in which a pyrolytic carbon layer is coated onto the filter.

### CROSS REFERENCE TO RELATED APPLICATIONS

This application claims the benefit of U.S. Provisional Patent Application Ser. No. 62/362,556, filed on Jul. 14, 2016 and entitled “BLOOD FILTRATION SYSTEM FOR IMPLANTABLE AND CLINICAL APPLICATION,” (Atty Docket No. 14172-701.100) and U.S. Provisional Patent Application Ser. No. 62/362,560, filed Jul. 14, 2016 and entitled “BIOCOMPATIBLE AND HEMOCOMPATIBLE MATERIAL AND FILTER,” (Atty Docket No. 14172-702.100), each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates to materials modified to have enhanced biocompatibility and hemodynamic properties for use in blood or biological fluid filtering and dialysis applications. This application relates to a medical device providing blood filtration for treatment of diseases such as end stage renal disease. The system uses hemofiltration and hemodialysis for treatment. The system includes a hemofilter, its casing, percutaneous and subcutaneous ports, external control components, external pump and fluid reservoirs. The invention relates to the treatment of renal failure and the replacement of a human kidney.

### BACKGROUND

The human kidney processes about 180 liters of blood every day and filters out around 2 liters of waste and extra water in the form of urine. The kidneys regulate the composition of the blood by removing waste products and excess water in blood plasma. Chronic kidney disease (CKD) is the loss of kidney function over a period ranging from months to years. Loss of kidney function can also affect other parts of the body and cause diseases such as heart failure. There is no cure for CKD but there are treatments available. Treatments manage to slow the progression of the disease, however, eventually complete kidney failure (end stage renal disease) may still occur in many patients. Renal replacement therapy aims to replace the kidney with a transplant of a donated kidney, dialysis. Hemodialysis and peritoneal dialysis (PD) involves long term ex vivo replacement therapy for support for renal function.

Majority of patients with end stage renal disease use conventional hemodialysis as renal replacement therapy. Conventional hemodialysis for end stage renal disease mimics the filtration function of the kidney. Dialysis procedures are normally carried out three times a week for three to five-hour sessions. Dialysis aims to emulate the function of the kidney by removing waste solutes and excess fluid from the patient's blood. Patients who go in for dialysis will have a high concentration of waste solutes in blood. Their blood is exposed to a semi-permeable membrane with a solute deficient dialysate. Solutes are removed by diffusing across the membrane and fluid is removed by pressure-driven ultrafiltration. Once the blood is purified it is returned to the patient.

Although hemodialysis removes small molecules well from the bloodstream, no current method has been established which provides selectively removing or retaining larger molecules. Dialysis solutions (called dialysate) must also be carefully controlled to ensure that their concentrations are adequate to ensure diffusion occurs across the membrane in contact with blood. About 120 liters of dialysate is used for each 4-hour session of dialysis.

Organ transplants is also difficult option as donors are limited and the need for the patient to take immunosuppressant medication that must be taken and the high risk of tissue rejection.

Wearable devices for kidney replacements work using similar technology to dialysis machines and offer more mobility and freedom for patients. Similar to dialysis procedures, devices such as the one described in Patent: EP2281591B1 use dialysate that is pumped across a semipermeable membrane to allow for molecules to diffuse out of the blood. This method however has the patient carrying a large device around the waist and is uncomfortable

Currently there are no implantable mechanical kidneys being used but there are many other patents such as U.S. Pat. No. 7,540,963B2 that uses silicon nano-filters and a bioreactor that contains human kidney tubule cells embedded within microscopic scaffolding. The silicon nano-filter uses ultrafiltration to filter out toxins, salts and some small molecules and water from the blood and the bioreactor uses a reabsorption system that returns water to blood to control blood volume.

Ceramic materials are defined as inorganic, nonmetallic solids composed of metals and nonmetals. Common ceramics have binary compositions such as metal or metalloid oxides, nitrides, and carbides. Depending on the composition of the ceramic, the material properties may widely vary, but in general, most ceramics are strong and brittle, display high thermal and electrical non-conductivity, and are chemically inert.

Ceramic materials have found novel applications in many areas including filtration techniques. Certain ceramic materials have a porous microstructure, in which the pores extend through the structure of the ceramic. These structures may vary widely, and include foams, honeycombs, fibres, hollow spheres, and interconnecting rods. The porous microstructure allows for separation and filtration applications ranging between ultrafiltration (>100 kD) to microfiltration (<100 kD).

Investigation into ceramic materials have also shown good biocompatibility properties, making this a promising material for human implants. However, ceramic materials have been shown to have poor hemocompatibility. Thus, for applications in which they have direct blood contact, clinical ceramic devices may pose a high risk of thrombosis.

As such, improvements are needed to adapt ceramic materials for use in devices contacting blood and in particular for filtering and separating components of blood. These improvements will also help in dialysis functions for blood or other bodily fluids.

### SUMMARY OF THE DISCLOSURE

In some aspects, a material is provided. The material comprises a ceramic substrate having an outer surface from which pores extend into said substrate; and a a coating over the surface layer(s) comprising a continuous layer of pyrolytic carbon which may infiltrate the substrate.

In some embodiments, the coating has a thickness of about 5 nm to 50 μm. The ceramic substrate can be a ceramic tube filter. The tube filter can comprise one or more channels. The ceramic substrate can be a ceramic disk filter. In some embodiments, said substrate is formed of a ceramic material selected from the group consisting of the nitrides, carbides, or oxides of aluminum, silicon, boron, titanium, zirconium, or mixtures thereof. The cut off for filtering molecules can be about 30 Da to 200,000 Da. The coating can provide greater biocompatibility and hemocompatibility than the unmodified ceramic substrate material. In some embodiments, the material is adapted and configured for use in a component or for integration within a housing or for positioning to filter human or animal blood as part of the improved operation of an implantable or external blood filtration system or a clinical or bedside blood filtration system. The material can be about 1 mm to 10 cm in width and 5 mm to 50 cm in length.

In some aspects, a method of manufacturing is provided. The method comprises providing a tube filter comprising a ceramic substrate with an outer surface from which pores extend into the substrate; mounting the tube filter between two mounting disks to form a mounted filter assembly; placing the mounted filter assembly in a quartz reactor; and pyrolizing a single layer of material comprising carbon on the ceramic substrate.

In some embodiments, the method comprises placing the quartz reactor in a tube furnace. In some embodiments, the mounting disks comprise a disk comprising an inner seat configured to seat an end of the ceramic tube filter; and a plurality of holes configured to allow passage of gas. The inner seat can comprise a hole through the disk. The pyrolizing can occur at temperatures between about 700° C. and 1200° C. In some embodiments, at least 40% of the pores remain open during and after the pyrolizing. The pyrolytic coating can be porous itself.

In some aspects, a hemofiltration device is provided. The device comprises an outer housing; an inlet port passing through the housing configured to receive a fluid; an outlet port passing through the housing to remove flow from the device; at least one ultrafiltration ceramic membrane inside the housing; an arterial inlet chamber configured to join to a patient's artery and to the inlet port; a venous outlet chamber configured to join to a patient's vein and to the outlet port; and a cap on each end of the housing configured to seal the device and distribute flow of blood evenly to both ultrafiltration ceramic membranes.

The housing can comprise a biocompatible material. In some embodiments, the housing comprises at least one of titanium, stainless steel, and PEEK. The patient's artery can be the iliac artery. The patient's vein can be the iliac vein. In some embodiments, at least one of the ultrafiltration ceramic membranes comprise tube filters. At least one of the ultrafiltration ceramic membranes can comprise a tube filter. In some embodiments, at least one of the ultrafiltration ceramic membranes comprises one or more channels. The device can comprise biocompatible tubing connected to each channel. In some embodiments, at least one of the arterial inlet chamber and the venous outlet chamber comprises a vascular graft. At least one of the caps can comprise a barb. The device can comprise sealing plates positioned near the caps. In some embodiments, the device comprises a dialysis port configured for connection with a percutaneous port. The device can comprise sealing O-rings at ends of the device. The membrane can comprise a coating. In some embodiments, the coating comprises at least one of a pyrolytic carbon and a diamond like carbon. The ceramic membrane can comprise a diameter of about 25 mm. The ceramic membrane can comprise a length of about 100 mm. In some embodiments, the ceramic membranes comprise a pore size of about 30 Daltons to 200,000 Daltons. The filter can comprise a filtration area of at least 0.1 m². The device can comprise a controller, valves and a pump on the outside of the patient connected to the device via a drive line. In some embodiments, the ceramic membranes are configured to hold a volume of about 200 ml. The device can be connected to renal artery and renal vein of a human kidney through dialysis port(s). In some embodiments, the device is connected to renal artery and renal vein of an animal kidney through dialysis port(s). In some embodiments, the device is connected to renal artery and renal vein of a human kidney through blood port (s). The device can be connected to renal artery and renal vein of an animal kidney through blood port (s). In some embodiments, the device is connected to another device through at least one of the blood port (s) or dialysis port (s) for further processing of the filtrate or blood. The device can be connected to another device through at least one of the blood port (s) or dialysis port (s), where the combination of the devices can purify blood without any need to use dialysate. In some embodiments, the device comprises two ultrafiltration ceramic membrane inside the housing. The device can be configured to concentrate uremic toxins in the filtrate and keep proteins such as albumin in blood.

In some aspects, a method for filtering blood is provided. The method comprises implanting a filtering device in a patient, the device comprising a housing; an inlet, an outlet, and two ultrafiltration ceramic membranes inside the housing; connecting an inlet of the device to an artery of the patient; and connecting an outlet of the device to a vein of the patient.

The method can comprise blood entering the device at about 1-2 psi. In some embodiments, the method comprises pumping dialysate to the device. The dialysate can be pumped at a pressure of about 0.5-15 psi.

### BRIEF DESCRIPTION OF THE DRAWINGS

The novel features of the invention are set forth with particularity in the claims that follow. A better understanding of the setup for making the invention will be obtained by reference to the following description that sets forth illustrative embodiments:

FIG. 1 illustrates an embodiment of a support disk for a tube filter substrate.

FIG. 2 depicts embodiments of support disks and a tube filter inside a quartz reactor (not to scale).

FIG. 3 shows an embodiment of a tube furnace setup for coating pyrolytic carbon on ceramic tube substrate.

FIG. 4 illustrates an embodiment of an alternate tube holders for coating the outside of tube filters.

FIGS. 5A-5B show scanning electron micrographs of the pyrolytic carbon coated filter.

FIG. 6 depicts an embodiment of a blood filtration device implanted within a patient.

FIGS. 7-9 show various perspective views of an embodiment of a blood filtration device.

FIG. 10 shows an embodiment of a blood filtration device with an upper portion of the housing removed.

FIGS. 11A-11C illustrate various views of embodiments of end plates of a blood filtration device.

FIGS. 12A-D depict various views of an embodiment of an inlet or outlet of a blood filtration device.

FIGS. 13A-D show various views of an embodiment of an O-Ring holder of a blood filtration device.

FIG. 14 shows an exploded perspective view of an embodiment of a blood filtration device.

FIG. 15 shows a graph comparing urea removal performance by the blood filtration device versus by dialysis.

### DETAILED DESCRIPTION

The present application describes the modification of ceramic filters in order to increase the biocompatibility and hemocompatibility.

The modification is a coating of pyrolytic carbon on the ceramic, while keeping the nanopores of the filter open. The ceramic can be used for filtration or dialysis applications to filter or dialyze blood or other biological fluids. The ceramic can include any and all of the nitrides, carbides and oxides of aluminum, silicon, boron, titanium and zirconium, or mixtures thereof.

The pyrolytic carbon is made by pyrolyzing a carbon containing compound. The pyrolysis occurs at temperatures between 700° C. and 1200° C. and can employ any carbon containing substance that is vapour in this temperature range. A carrier gas can be used along with the carbon containing substance but is not necessary. Small hydrocarbon compounds such as methane, ethane, propane, hexane, acetylene, ethylene, benzene, etc., are most suited to this application, but are by no means the only substances.

The filters can be considered as any solid material having a porous structure with pores on the order of about 10 Å to 100,000 Å. The solid could be comprised of a single piece of material or the combining of nanoparticles or microparticles to form a single structure.

In some embodiments, the ceramic may be in a tubular shape with porous walls such that the biological fluid runs through the inside and the filtrate comes out through the walls of the tube. The tube may have one or many channels for the fluid to pass through. In other embodiments, the filters can be disk shaped with blood or biological fluid running on one side and the filtrate or dialysate on the other side.

The biocompatibility of an object is directly related to its form, roughness and the material of the area in contact with bodily fluids. These properties can be under stricter restrictions when in the presence of blood due to the many clotting factors and proteins in the blood that adhere to foreign objects. Therefore, achieving 100% biocompatibility does not assure 100% hemocompatibility. In either case, there are very few materials that the body doesn't reject and even fewer that have the mechanical properties required for long term use. Carbon is one of these materials that exhibits good hemocompatibility and can be made to have the right mechanical properties based on the allotropes used. Pyrolytic carbon is a form of graphitic carbon that is highly resistant to thrombus formation and so widely employed for use in long-term medical device coating.

In the current application, pyrolytic carbon is coated on ceramic filter to increase the biocompatibility and hemocompatibility. The layer of pyrolytic carbon is from 5 nm to 50 μm which varies depending on the final filter pore size required. This layer serves two purposes. Firstly, the pyrolytic carbon is very thrombus resistant so clotting does not occur easily. Secondly, the thin layer helps to smooth out the surface thereby decreasing the surface roughness and increasing biocompatibility further.

Ceramic filters are available in different shapes, sizes and pore sizes. For most filtration applications, disks and tube are the most common shapes used. Size is dependent on the application; though, for most biological application, the size ranges from 10-90 mm diameter disks and 10-50 mm diameter, 100-250 mm long tubes. Ceramic disk filters are commercially available from vendors such as Sterlitech, Superior Technical Ceramics, Outotec, etc. Single- and multi-channel ceramic tube filter membranes are commercially available from the vendor Atech Innovations, Tami Industries, Pall, lnopor, etc. In their current industrial form, these commercial grade materials are unsuited for the filter applications described herein. However, various embodiments of the techniques described herein may be utilized advantageously to modify the material properties of the ceramic material using one or more additional processing steps as needed and described herein.

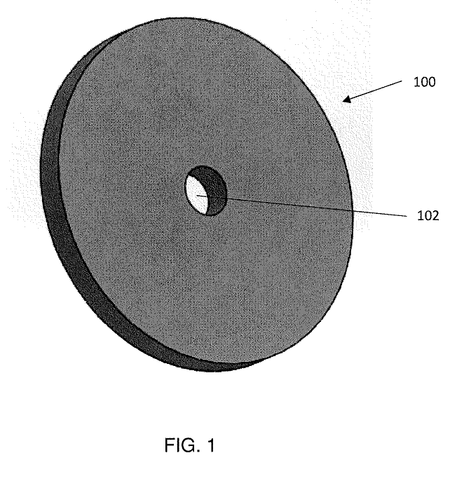

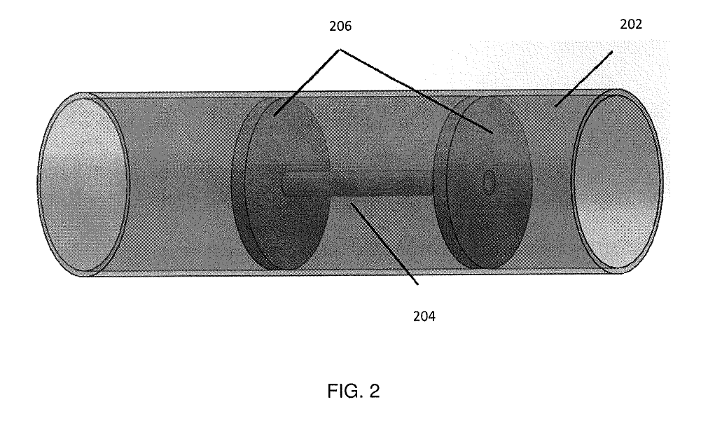

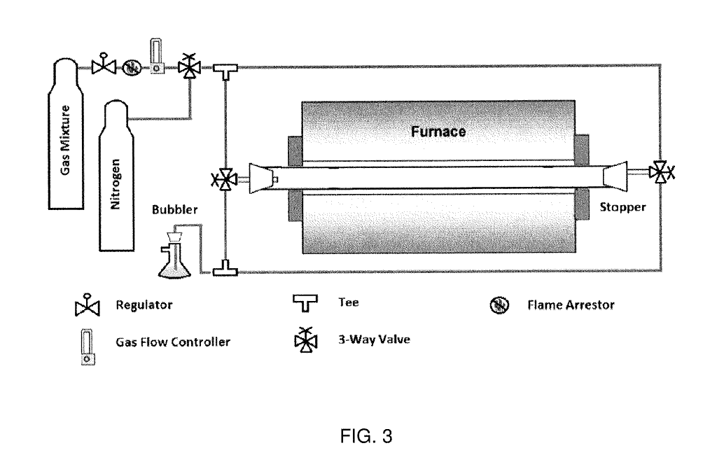

In some embodiments, ceramic tube filters are obtained that are in smaller diameter than the quartz reactor in which they will be coated. Ceramic membrane filters are received as either single or multi channel tubes, with porous microstructured ceramic walls. The diameter of the tube and the inner channels may vary depending on the number of inner channels. The filter is prepared for pyrolytic carbon coating via mounting on two steel disk holders, approximately the same diameter as the quartz reactor (See FIG. 1). The disks can be made out of any material able to withstand the temperature at which pyrolysis is occurring. Steel is suggested due to the high melting point and relatively cheap cost. Each disk 100 has a hole 102 drilled out of the centre, relatively the same diameter as the tube filters. As shown in FIG. 2, the entire 3-component setup, comprising the tube filter 204 and the support disks 206 is placed into a quartz reactor. The quartz reactor is then placed into a high temperature tube furnace (See FIG. 3) for coating of the ceramic tube substrate with pyrolytic carbon. In other alternatives, components above are modified to provide an appropriate reactor shape, size and configuration suited to the size, shape characteristics and type of ceramic membrane being processed.

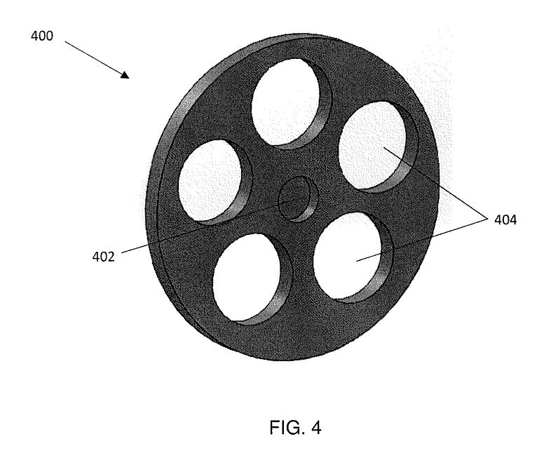

In other embodiments, the methods and techniques described herein may be adapted to provide inventive coating on the outside of the tube to enhance its bio/hemocompatible qualities or characteristics. In such cases, the holders 400 can be modified such that there is an inner seat 402 for the tube to sit inside while large holes 404 in the rest of the disk holder allow the passage of gas (see FIG. 4). If both the inside and outside of the tube is meant to be coated, then the central hole 402 could be drilled through.

In another embodiment, disk filters may need to be made bio/hemocompatible. In this case, disks that are slightly smaller than the diameter of the quartz reactor can be put inside the reactor as is, or on top of a steel plate/disk.

The reactor is setup such that gas can be introduced from either end of the quartz reactor and then exit from the opposite end. This can be switched around so that an even coating of the pyrolytic carbon can be deposited along the entire length of the tube.

The filter is heated in a furnace in an inert atmosphere at a rate of 5-10° C./minute until reaching the temperature of coating. This is held for 15-20 minutes for the temperature to be more uniform within the reactor. The carbon-containing gas is then introduced with or without a carrier gas. Pyrolysis occurs as the gas reaches the hottest parts of the reactor and the atomized carbon deposits onto the surface of the filter. The temperature and gas inflow is held for 1-6 hours.

In one specific aspect, half way through the planned time for pyrolysis, the direction of the gas inflow is switched to the other side of the reactor. In other embodiments, the reactor is operated to reverse the flow multiple times during the coating process. In other embodiments a computer controller is used to control the operating environment of the furnace including temperature, gas flow rates, ramp-up, ramped down cycles and the like.

After the coating cycle is complete, the furnace is ramped down at a rate <5° C./minute to 500° C. in order to prevent thermal cracking. In other aspects or optionally, further ramping down can occur at a number of different rates.

Before removal from the furnace, the filter is treated in the furnace at ambient pressure in a nitrogen gas atmosphere.

At least two types of gases are needed for the pyrolysis: an inert gas and a carbon-containing compound. The inert gas is used to purge the reactor while heating or before the carbon-containing gas is introduced. If there is oxygen left in the reactor, the carbon would oxidize and carbonization would not occur. If the substrate is stable in air at high temperature, the inert gas purging can happen right before introduction of the carbon containing gas. Purging can also be done as the temperature is ramping up. Purging should be done with reversing the flow of gas as well so that the entire system contains no oxygen.

After purging is complete, the carbon containing gas is introduced. This gas can be a pure source or a mixture, though the mixture should have >10% of the carbon containing compound (by volume) so that sufficient pyrolysis can occur without leaving the system running for many hours. The carrier gas, if a mixture is used, should be inert so that side reactions are minimized.

The ideal gas flow rate can be between 100-1000 mL/min, with larger flow rates being used for larger surface areas and larger reactor volumes. Lower flow rates can be used but coating duration will be longer unless the pressure is increased or the reactor volume is small.

To confirm uniformity of the carbon coating, electrical impedance methods can be employed. A measure of electrical resistivity is taken across a fractional length of the filter over multiple areas of the coating surface. Since the carbon coating is conductive, the thicker the coating, the lower the electrical resistance. Hence, variations in the resistance at different areas of the filter indicates fluctuations in the coating uniformity.

Adhesion of the coating can also be determined using electrical impedance methods. Distilled water is flowed through the tube filters (or across disk filters), whereupon unadhered carbon is removed. This causes a change in the resistivity, which can be measured before and after subjecting the filter to water flow. Good adhesion of the carbon coating is indicated by no change in resistivity, while poor adhesion is indicated by an increase in electrical resistivity.

To confirm filter operation and hemocompatibility, distilled water can be flowed through each of the tube filters (or across the disk filters) and the flux is measured. Pig blood, obtained from a butchery, can also also pumped through a coated and uncoated filter. Platelet adhesion can be measured using a differential platelet count on the blood pre- and post-filtration. This is used as a marker for the hemocompatibility with a lower differential showing better compatibility.

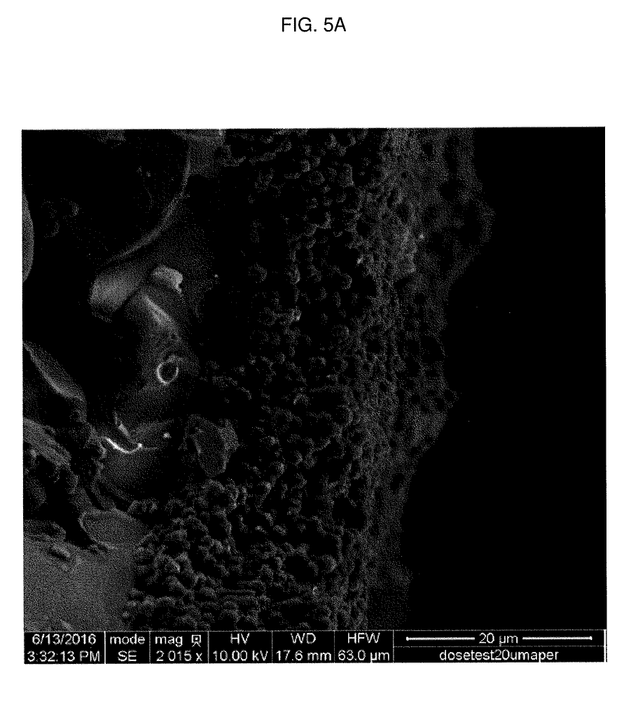

FIGS. 5A-5B show a scanning electron micrograph of a nanofilter coated by pyrolytic carbon. As shown in FIG. 5B, the filter has 3 layers. When used in the blood filtration application, blood is in contact with the pyrolytic carbon layer. The pyrolytic carbon cating comprises pyrolytic carbon spheres formed and melted together under high temperature. This layer can have two jobs. Pyrolytic carbon has excellent hemocompatibility properties and is in use in blood contacting surfaces of devices such as heart valves and left ventricular assist device (LVAD). The space between spheres act as a porous structure (mesh) blocking passage of white blood cells, red blood cells and platelets, but allows passages of plasma. All uremic toxins have a molecular weight of smaller than 60,000 Da. Therefore, the filtrate contains all uremic toxins as well.

The middle layer is a nano filtration layer which is a porous ceramic structure comprising a combination of at least one of zirconium oxide and/or titanium oxide with pore sizes <10 nm. This layer filters out proteins such as albumin (MW: 66,500 Daltons) from the plasma that has passed the pyrolytic carbon layer. The filtrate which has passed this layer would have minimal or zero amounts of albumin. This layer is also hemocompatible and blocks passage of at least 90% of blood component larger than 60,000 Da

The third layer is a microporous ceramic support structure comprising a combination of at least zirconium oxide and/or titanium oxide. This layer is hemocompatible and acts as a support for other layers of the nanofilter and maintains the nanofilter's integrity. This layer is porous with pore sizes of more than 100 nm.

Exemplary previously attempted coating processes are described in Li, Yuan-Yao, Tsuyoshi Nomura, Akiyoshi Sakoda, and Motoyuki Suzuki. “Fabrication of Carbon Coated Ceramic Membranes by Pyrolysis of Methane Using a Modified Chemical Vapor Deposition Apparatus.” Journal of Membrane Science 197.1-2 (2002): 23-35. Patent: U.S. Pat. No. 3,471,314 A—Pyrolytic Carbon Coating Process, the contents of each of which is incorporated by reference in its entirety for all purposes.

In the paper mentioned above, two filters with pore sizes 100 nm and 2.3 μm were coated by pyrolytic carbon. Also, it is mentioned that the pores are narrowed down due to pyrolytic carbon coating layer added. However, the current technique utilizes much smaller pore sizes. As described herein, some embodiments comprise (i) pyrolytic carbon coating of ceramic filters with pores smaller than 10 nm and (ii) maintaining of the filtration properties of the substrate with <10 nm pores during and after pyrolytic carbon coating. In particular, generally, the shape and size of the pores <10 nm can change under high temperature processes needed for the pyrolytic carbon coating. However, the current technique maintains the pore size in the coated filter in the same range as what was before coating.

The filter material provided by the processes described herein may be used in a number of different embodiments depending on the system where the filter will be used. A few embodiments are mentioned but are not an exhaustive list of the uses of these filters nor of the variety of size, shaped over all geometry used in any of a number of various alternative embodiments. The form factor of the filter for any specific embodiment depends on and is responsive to a number of design considerations for where the filter will be employed and the overall characteristics of the filter system.

In still other aspects and alternatives, the treated material may be modified, sized, shaped, incorporated into a form factor or a component or components to accommodate the casing or design of a pre-existing system or a filter material adapted and configured to have a form factor for use in a system or method described in any of the following references, each of which is incorporated by reference in its entirety: WO2010088579A2; U.S. Pat. No. 7,540,963B2; US20090131858A1; WO2008086477A1; US20060213836A1; U.S. Pat. No. 7,048,856B2; US20040124147A1; US20120310136A1; WO2010088579A2; U.S. Pat. No. 7,540,963B2; US20090131858A1; U.S. Pat. No. 7,332,330B2; US20060213836A1; U.S. Pat. No. 7,048,856B2; US20040124147A1; WO2004024300A1; WO2003022125A2; US20030050622A1; WO2010057015A1; US20100112062A1; US20040167634A1; WO1998009582A1; U.S. Pat. No. 9,301,925B2; US20160002603A1; US20130344599A1; US20090202977A1; WO2007025233A1; US20120289881; US20130109088A1; U.S. Pat. No. 8,470,520B2; WO2013158283A1; U.S. Pat. No. 7,083,653; Nissenson A. R. a, Ronco C. b, Pergamit G. c, Edelstein M. c, Watts R. c; “The Human Nephron Filter: Toward a Continuously Functioning, Implantable Artificial Nephron System”; Blood Purif 2005; 23:269-274; (DOI:10.1159/000085882); Jeremy J Song, Jacques P Guyette, Sarah E Gilpin, Gabriel Gonzalez, Joseph P Vacanti & Harald C Ott; “Regeneration and experimental orthotopic transplantation of a bioengineered kidney”, Nature Medicine, 19, 646-651; (2013); doi:10.1038/nm.3154; Madariaga M L, Ott H C., “Bioengineering kidneys for transplantation”, Semin Nephrol. 2014 July; 34(4):384-93. doi: 10.1016/j.semnephro1.2014.06.005. Epub 2014 Jun. 13; Song J J, Guyette J P, Gilpin S E, Gonzalez G, Vacanti J P, Ott H C., Regeneration and experimental orthotopic transplantation of a bioengineered kidney. Nat Med. 2013 May; 19(5):646-51. doi: 10.1038/nm.3154. Epub 2013 Apr. 14. In still another aspect, any of the above described systems or components described therein is modified using one or more of the techniques described herein or is replaced with a compatibly shaped and sized component having the optimized characteristics described herein for use in implanted or clinical systems that contact flowing blood within a human or animal body.

In still further optional or alternative embodiments, there are methods for performing post processing steps to cut or mold the treated component or filter or material into desired shape along with or alternatively positioning the filter material into a suitable frame or within a housing of a specific filtering system.

In another aspect, the filter material provided by the processes described herein may be used in a number of different embodiments depending on the system where the filter will be used. The form factor of the filter component depends on a number of design considerations for how the filter will be employed and the overall characteristics of the filter system. In one aspect, the filter material may be in a final shape for use in a filter housing without a frame. In another aspect, the filter material may be cut, shaped, sized for use in an edge frame or a frame holder within or along the casing that is adapted and configured to engage with or received by the housing. In still another aspect, the filter material may be placed within a support frame that includes a shape, webbing, openings, apertures, indentations, or other features that will secure the filter material within the frame. The frame then includes various features or characteristics that then engage with a portion of the filter component or another housing of the filter system so that the filter material is positioned within the flow path of the filtering system.

Additional aspects of an embodiment of the invention is further illustrated by the following non-limiting example(s)

### EXAMPLES

### Example 1

A sample ceramic substrate was obtained from Atech Innovations in the form of a single channel tube-shaped alumina filter, with full outer diameter of 10 mm and inner channel diameter of 6 mm. The filter surface per element for the unaltered filter is 0.01910.023 m². The tube filter had a macroporous structure with a pore size >10 microns, and an inner, microporous structured layer with effective pore size of 0.8 microns.

Two steel support disks of ¼ inch thickness and approximate diameter of 75 mm, with a 10 mm diameter hole drilled through the center (see FIG. 1), were placed at each end of the tube substrate, so that each end of the tube fit into the holes. This assembly was placed into a high temperature tube furnace, inside of a quartz tube reactor that was 5 feet long and had an inner diameter of 75 mm (see FIG. 2). The reactor was sealed using black rubber stoppers on each end, and subsequently purged of oxygen by flowing nitrogen through one end. The system was set up such that gas could be introduced into the reactor from either end by switching the direction of a few 3-way valves.

The substrate was heated at a rate of 10° C./min until it reached 1000° C. in a nitrogen atmosphere. Nitrogen was flowed through the reactor for 10 minutes, before switching direction of the flow and purging for another 10 minutes. A mixture of 80% nitrogen and 20% methane was introduced into the reactor for 2 hours, switching direction of the gas flow halfway through. The reactor was then cooled down to 500° C. at a rate of 5° C./min under nitrogen gas flow, followed by air cooling to room temperature under no gas flow.

Coating adhesion was checked by electrical resistivity methods. A measurement of electrical resistivity is taken across the inner coating of the tube. Water was pumped at <3 psi through the inner channel for between 1 and 4 hours. Once dried, the resistivity was measured again, with minimal change indicating good adhesion.

Hemocompatibility was checked immersing a coated and uncoated substrate into separate baths of fresh pig blood. The pig blood was obtained from a butcher post butchery, and mixed with 10% EDTA as anticoagulant (using standard 1.5 mg/mL of blood). Pig blood samples pre and post immersion were sent to Antech Diagnostics for a complete blood count. Platelet counts showed a >3× decrease in platelets lost from a coated substrate compared to an uncoated substrate.

### Blood Filtration System for Clinical Applications

There are over 650,000 patients with end stage renal disease in the United States and there are only 20,000 kidneys available for transplantation each year. The demand for a kidney is so high and the number of donors is so low that patients sometimes have to wait 5-7 years for a kidney transplant. The only option they have to use in those years to survive is dialysis.

Dialysis was invented by Dr. Kolff in 1943 and it has been saving many lives since then. However, the technology has not changed much for decades. Currently, dialysis patients are generally connected to a large dialysis machine watching their blood in circulation in a plastic tube, 3 times a week for 4 hours each time with not much hope for any change in the near future. Such patients suffer emotionally and physically and they are in pain. In fact, the mortality rate of patients under dialysis is 65% in 5 years and the process is very costly. Dialysis costs about $82,000 per patients per year and that makes dialysis a huge market. The dialysis market was valued at $70 Billion in 2015 and is estimated to grow to $100 Billion by 2020.

The current application discloses a unique, implantable, nano filtration technology that mimics the filtration property of kidneys and is very blood friendly. The nano filters disclosed herein can be so efficient that they function based on normal blood pressure. This technology can provide renal replacement therapy continuously and automatically at all times and that provides freedom and a more normal life for dialysis patients

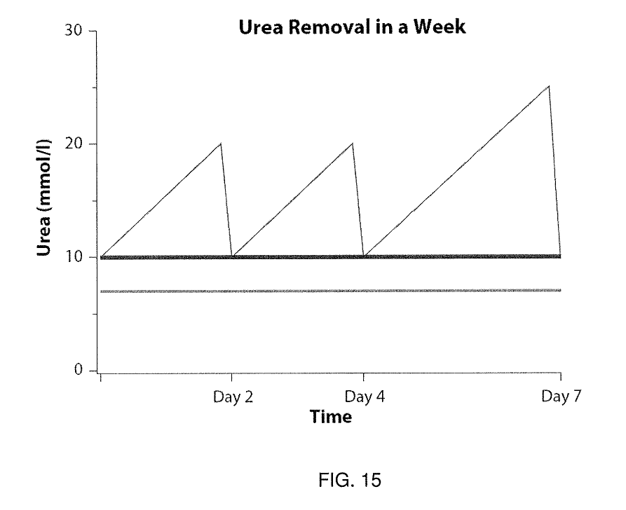

Dialysis patients have high level of uremic toxin and excess water in their blood. In fact, the level of uremic toxins and water in their blood peaks three times a week, right before the dialysis session. The maximum peak is usually after the weekends or holidays. The filters and devices described herein can function to maintain the level of uremic toxin and excess water in the body of patients at the normal and safe level at all time, as shown in FIG. 15. Clinical testing has shown that the device is able to remove fluid and solutes from the animal's blood in pig animal models.

The present application discloses a device that has a blood inlet and a blood outlet that are connected to an artery and vein respectively. The inlet draws blood into a chamber that distributes the blood into at least one tubular filter. In the current device, two tubular filters (e.g., filters described above with respect to FIGS. 1-5) are used. The filters use ultrafiltration to remove waste products and excess water from the blood. A vascular graft connects blood inlet to an artery and another vascular graft connects the blood outlet to the vein.

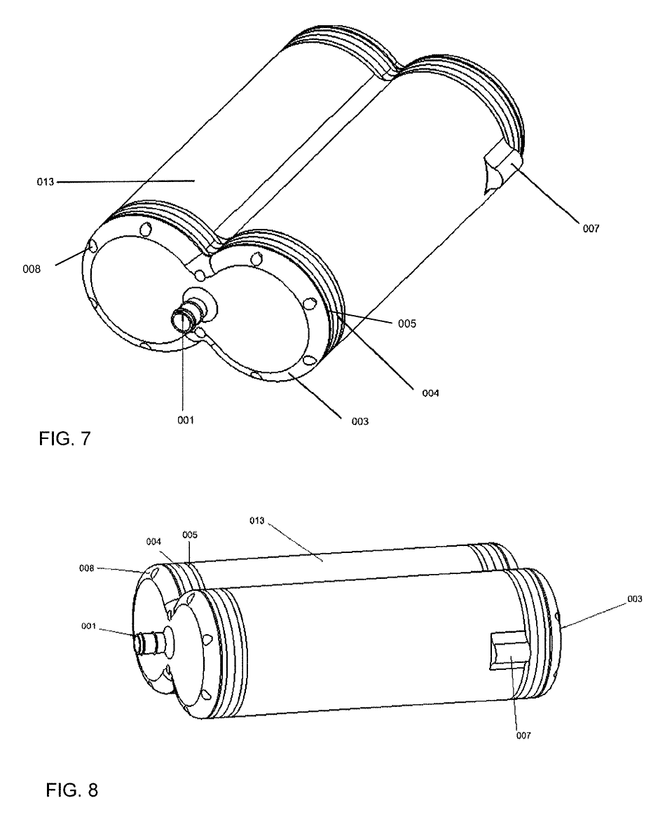

Ultrafiltration is a membrane based filtration process. Filters for the present invention employ the use of ultrafiltration and are used to filter out excess water, uremic toxins, and excess minerals from blood. In some embodiments, a ceramic tubular filter 009 (FIG. 10), is used as the membrane for the ultrafiltration.

Blood is separated from the system and sent to the renal vein while the waste is sent to the bladder.

The interior chamber holds and seals the filters with the help of two end plates on each side. It also consists of two small external ports that allow for dialysis to be pumped into the housing. O-rings and gaskets allow for the device to be sealed.

Dialysis solution can be pumped into the interior chamber percutaneously using an external pump. This allows for dialysis solution to come in contact with the exterior of the tubular filters. Valves and a controller regulate the flow and pressure of the dialysis solution. This allows for the dialysis to permeate the filters and for ion exchange to occur.

The device casing is constructed using biocompatible grade materials such as titanium, stainless steel or PEEK. The filters are coated with a biocompatible coating such as zirconium oxide, pyrolytic carbon or diamond like carbon (DLC). Fittings and screws are also from bio-compatible materials such as medical grade stainless steel or titanium. The tubing and rubber are made from medical grade materials such as PTFE, silicon and tygon.

In some embodiments, the device comprises biocompatible tubing going into each membrane channel. The tubing would loop in and out of the filter at each membrane. These loops can help ensure that each membrane would receive the maximum amount of blood to ensure proper ultrafiltration. It would also ensure that blood would not be exposed to any impact force or unnecessary turbulent flow.

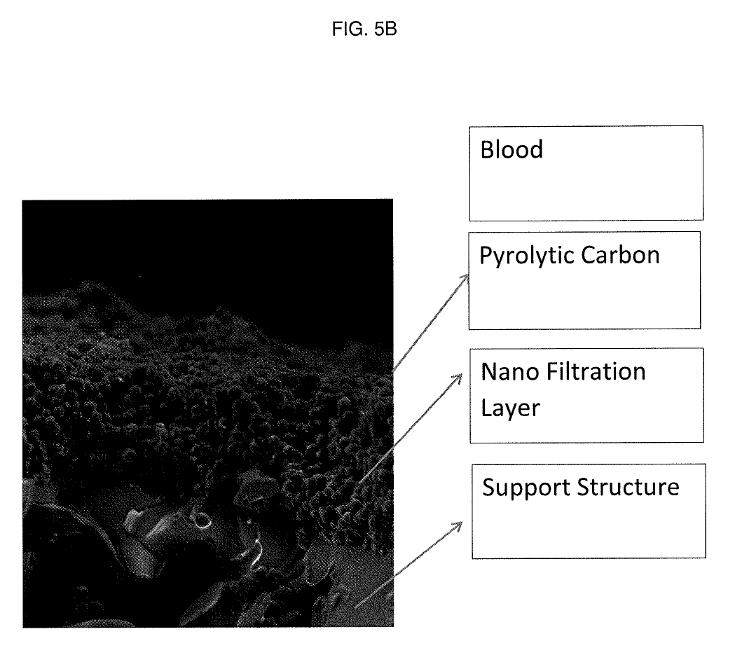

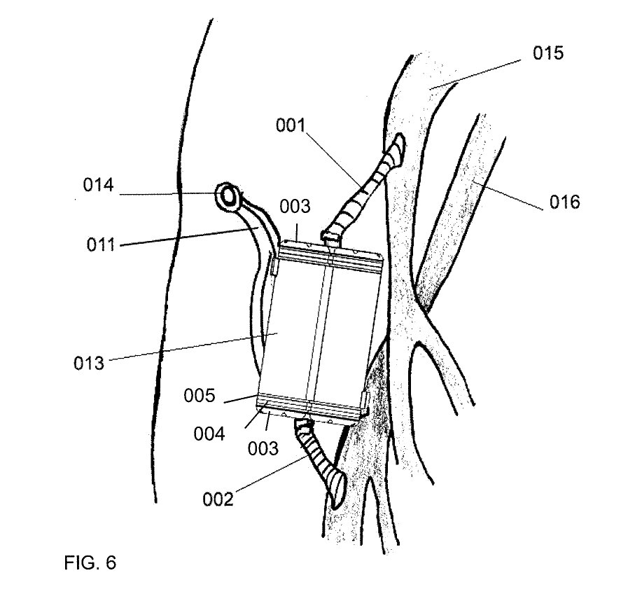

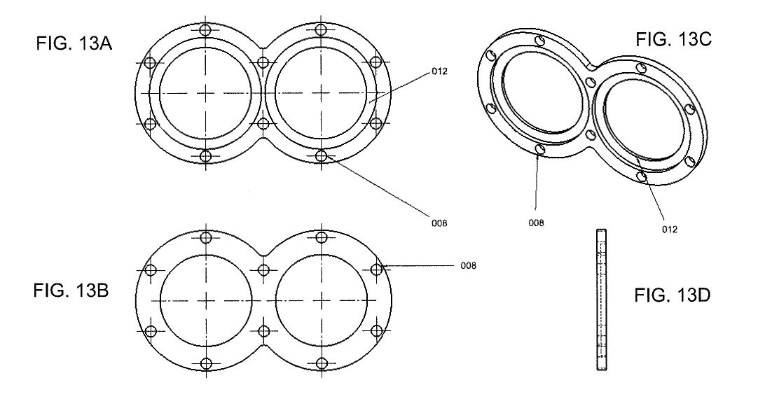

The present invention utilizes ultrafiltration and hemodialysis to replicate a human kidney's function. The device utilizes two multichannel tube filters to remove filtrate from the blood. The filtrate contains blood components such as water, electrolytes and uremic toxins, proteins. Also, with the help of dialysate the device can remove more solutes from the blood. The device comprises an outer housing 013 that acts as a collection area for the ultrafiltrate, an area where dialysis can occur and as a holder for the filters. FIG. 6 shows the implanted location and connections of the entire device near the iliac artery 015 and the iliac vein 016. The outer housing 013 is connected at each end by a pair of plates 004, 005 (FIGS. 7, 8) that both hold and seal the device. The device is sealed using hemocompatible o-rings and gaskets made out of silicone and tygone and positioned at location 012 (FIG. 13). The plates expose the faces of each filter to blood on both sides of the housing. They are shaped to equally distribute blood to multiple channels on the filters. In some embodiments, the blood inlet 001 can be connected to each channel in the ceramic filter through a blood distribution piece. In the blood distribution piece, blood enters through blood inlet 001 and distributes into small tubes, each connected to one filter channel. Blood enters and leaves the system at the inlet and outlet caps 003. These caps 003 are located each end of the housing 013 on top of the sealing plates. Both the inlet and outlet are connected to vascular grafts which allows blood to enter and leave the system. Graft 001 is connected to the inlet and graft 002 is connected to the outlet. The ends of the caps can be barbed to allow for the grafts to grip on and be secured. Blood can enter the system at a pressure of 1 to 2 psi.

The casing 013 can comprise medical grade 5 titanium. Titanium has a high strength, low weight and has a high corrosion resistance. It is commonly used in implantable applications such as joint replacement, spinal screws, and implantable devices. Other materials (e.g., stainless steel) are also possible. In some embodiments titanium is preferred over stainless steel due to its higher strength to weight ratio.

FIGS. 7-9 show top, side, and front perspective views, respectively, of the device. FIGS. 7-9 show the exterior housing 013 and plates 004, 005 located at the ends of the housing. Cap 003 is shown at an end of the device. The cap 003 includes holes 008 which can be used to screw and seal the cap 003 to the body 013. A portion of inlet graft 001 is shown at the inlet end of the device. In the views of FIGS. 7-9, dialysis port 007 is also visible.

FIG. 10 shows a front view of the device with the top half of the housing 013 removed, allowing visualization of the filters. Blood enters the tubular membranes at one of the membrane faces 009. These membranes are available in different shapes, sizes and pore sizes. For example, the pore size can have a cut off value between 30 Da to 900 kDa. The membranes can be made from materials comprising zirconium oxide, TiO₂ or AlO₂. Other materials are also possible. To ensure the body will accept the filters, they can be coated with a biocompatible material such as pyrolytic carbon or a diamond like carbon. In some embodiments, the filter comprises a multichannel tubular filter. This filter configuration can advantageously maximize the active filtration area. The filters can have a diameter of about 20-30 mm. The filters can have a length of about 5-500 mm. The pore size can be about 30 Daltons to 200,000 Daltons. The active filtration area can be about 0.075-2.5 m². In some embodiments, the filters have a 25 mm diameter; a length of 100 mm; a pore size of 50,000 Daltons; and an active filtration area of 0.1 m². In some embodiments, the number of channels can vary as long the filter has as a filtration area of 0.1 m\hat{ }2 and a pore size of 50,000 Daltons. This pore size allows keeping most of the albumin in blood, while removing water, solutes smaller than 50,000 Daltons, urea, and creatinine.

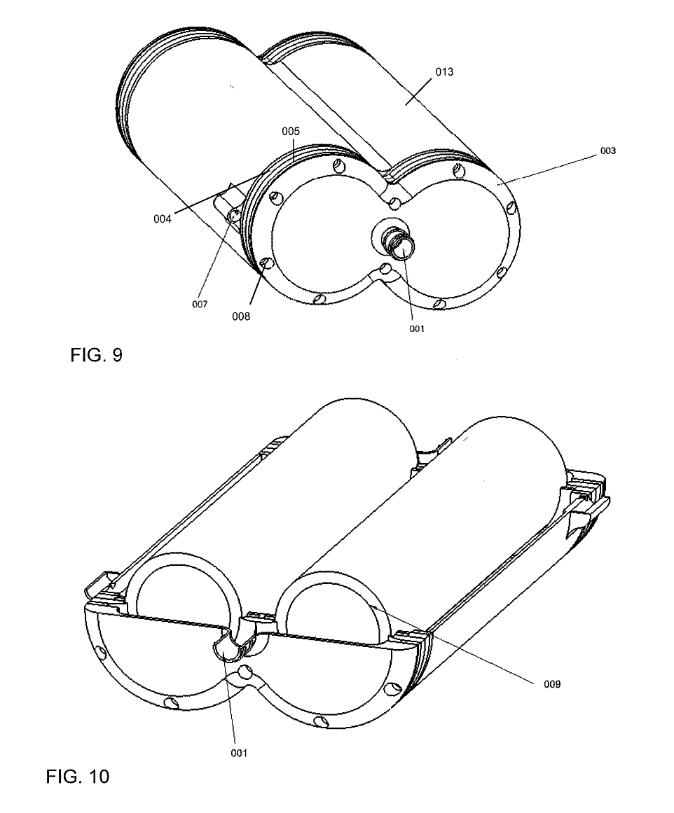

FIGS. 11A-11C show front, back, and back perspective views, respectively of an embodiment of end plates 004. The end plates 004 comprise a surface 010 that is configured to distribute blood to the filter. Apertures 008 are shown that allow end plates 004 to be sealed to caps 003 and the housing 013.

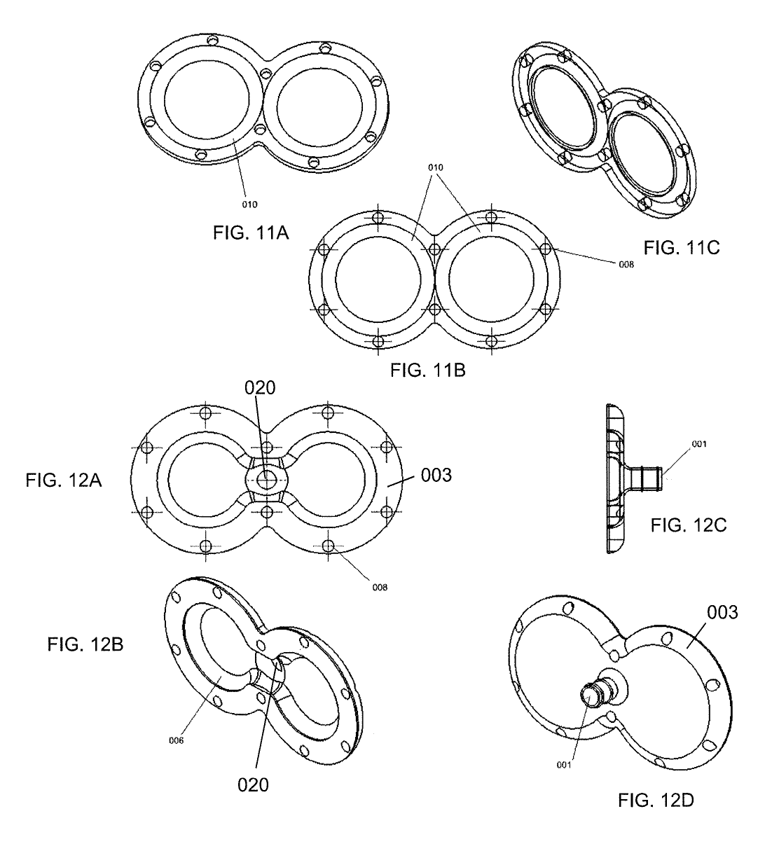

FIGS. 12A-12D show front, back perspective, side, and front perspective views of the area around the inlet 001. The outlet may have a similar configuration as that shown in FIGS. 12A-12D. FIGS. 12A and 12B show the inlet 020. As shown in FIG. 12B s a tapered surface 006 can function as a funnel within the cap 003 that holds blood received through the inlet 001 or awaiting exit through the outlet. FIGS. 12C and 12D show that the cap 003 has a rounded shape, providing atraumatic surfaces for implantation and reducing risk of thrombus. Screw apertures 008 can extend through cap 003, as described herein. Vascular graft 001 can be connected to the inlet 020 or the outlet.

FIGS. 13A-13D show back, front, back perspective and side views of embodiments of the end plates 005. A recessed portion 012 of the end plate 005 is configured to seat an O-Ring (not shown) for sealing the ends of the device.

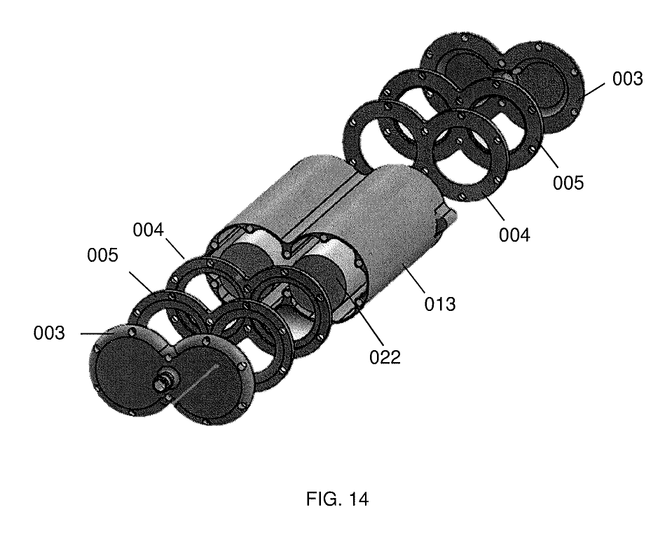

As shown in FIG. 14, the end plates 004, 005 and the cap 003 can have a sandwich construction at ends of the housing 013 of the device. FIG. 14 also shows filters 022 within the housing 013. The cap 003 is positioned at an end of the device. End plate 005 is positioned inside the cap. End plate 004 is positioned inside end plate 005. Order of these components may be modified in some embodiments. Additionally, in some embodiments, features of the components (e.g., funnel, O-Ring seat, etc.) may be differently distributed between the components.

On the outside of the patient's body a controller, pump and valves will be present to regulate the intake of dialysate. A flow rate of 100-800 mL/min with a variable pressure allows the device to simulate dialysis treatment used in dialysis machines.

Dialysis solution is pumped through silicon tubing to the system at a pressure slightly higher than that of the iliac artery. The pressure can range from about 0.5 to 15 psi. These parameters can help ensure the dialysis solution just barely permeates the membrane to ensure ion exchange occurs. Pressure is then lowered and dialysis solution is removed from the system. This system will remove solutes from blood. Dialysate can enter the device via percutaneous port 014 that will exit the patient's body. The external pump can also be used for cleaning the filters. In some embodiments, the time between dialysate entering the device and exiting the device can be a few seconds (e.g., 2-3 seconds, 1-5 seconds, 1-10 seconds, greater than 10 seconds, etc.).

The device is sutured to the patient's posterior body wall using four attachments that are present on the device and are placed on the body of the casing 013.

The whole device can have a length of about 85-135 mm. The device can have a width of about 50-90 mm. The device can have a height of about 25-55 mm. In some embodiments, the device dimensions are about 107×70×38.5 mm. The vascular grafts positioned at either end of the device can be about 5-7 mm. In some emboidments, the grafts are about 6 mm, and are attached to each end of the device using clamps. The grafts can sit on barbs positioned on the cap and the clamp can sit on the graft and hold it to the barb. The device can comprise titanium fittings at the dialysis ports and biocompatible silicon tubing to pump dialysate into the system. The filter in the device uses a volume of approximately 200 ml of blood to fill up.

Data from animal blood testing is shown in Table 1 below. A filter according to this application was used for in vitro filtration of animal blood.

TABLE 1

| | Blood | | Filtrate | |
|---|---|---|---|---|
| GLU | 45 | mg/dl | 76 | mg/dl |
| BUN | 29 | mg/dl | 42 | mg/dl |
| CA | 9.9 | mg/dl | <4.0 | mg/dl |
| CRE | 0.6 | mg/dl | 0.8 | mg/dl |
| ALB | 3.5 | g/dl | 0.0 | G/ul |
| PHOS | 7.9 | mg/dl | | Mg/dl |
| NA+ | 143 | ol/l | >180 | mmol/l |
| K+ | 5.5 | mmol/l | 7.6 | mmol/l |
| CL− | 102 | mmol/l | >140 | mmol/l |
| TCO2 | 22 | mmol/l | 28 | mmol/l |

The results show that the filter described herein can concentrate uremic toxins in the filtrate and keep proteins such as albumin in the blood.

Table 2 below shows additional testing of a pyrolytic carbon filter according to this application tested in a pig animal model with no kidney function. A nephroctomy was performed on the pig model before the device was attached to the animal.

| | | |
|---|---|---|
| ALB | <1.0 | g/dl |
| ALP | <5 | u/l |
| ALT | 10 | u/l |
| ANY | 353 | u/l |
| TBIL | 0.3 | mg/dl |
| BUN | 8 | mg/dl |
| CA | <4.0 | mg/dl |
| PHOS | 5.9 | mg/dl |
| CRE | 1.2 | mg/dl |
| GLU | 14 | mg/dl |
| NA+ | 254 | mmol/l |
| K+ | 5.3 | mmol/l |
| TP | <2.0 | g/dl |
| GLOB | — | g/dl |

The collected sample contains a minimum level of albumin. Additionally, the presence of uremic toxins (urea and creatinine) in the filtrate sample is confirmed.

In one alternative embodiment, the filter used in an embodiment of a system illustrated and described in FIGS. 6-13 may be one configured according to one of the embodiments described with respect to FIGS. 1-5. In still other aspects, a number of different form factors of the filter and/or other components of the system as in FIGS. 6-13 may be providing according to variations particularly as they relate to the manner the filter design and material may be used, configured or adapted for a particular use based on a particular filter design or, optionally, for a filter used in any of the other filter systems described herein.

The filter system described herein may be adapted to a number of different clinical and implanted configurations. For example, in an implantable version of the filter system, there may be embodiments that are fully implantable or partially implantable. In some embodiments, some of the components or functions of the system may remain outside of the patient's body but within communication with the implanted device using any suitable transcutaneous communication modality. In still other aspects, a battery in the implanted portion may be charged transcutaneously. In still another aspect, there are control modules that operate in concert in terms of functionality performed by each in terms of controlling, reporting, updating or modifying control software or data streams used between external and internal components of the system or in the communications between the system and outside sources such as remote computer systems such as cloud computing systems. As a result, an operating system or controller scheme used for the operation of the system may be performed in a number of suitable ways.

While one exemplary surgical implantation site is illustrated in FIG. 6, other possible implantation sites are possible based on patient anatomy, disease state and other clinical or surgical factors. In one aspect, features of a system adapted for implantation into a patient with impaired or compromised kidney function or aspects of the method of surgical implantation or features can be adapted given due consideration to the future plan for the patient (e.g., to receive a transplant kidney, for use with a patient in need of an artificial kidney, perhaps for an extended term). In this aspect, the implantation site or design factors of an embodiment of the device may be modified based on specific details of the anatomical site and clinical use for the kidney failure patient and those activities that are related to the period while the patient is waiting for a donor. In another aspect, there may be modification to one or more aspects of the implantable components or the surgical plan to modify or adapt to the positioning of the artificial kidney in relation to a natural kidney, to a diseased or impaired kidney, to other anatomical or physical impairments in the patient, including as well the position, location and placement of inlets, outlets, the input and the output of the artificial kidney and the like are connected to the patient's vasculature, the patient physiology and other considerations of the patient implantation procedure and then subsequent ease of use for the implantable unit by the patient.

Still further to the implantation process, there are still other form factors possible in various other embodiments whereby the overall form factor of the implantable kidney takes into account a number of different considerations including, for example, the implantation site, orientation and connection points of the artificial kidney in relationship to the natural kidney or transplanted kidney and the surgical site of a partially removed or fully removed kidney. In each of these different clinical cases, there may be provided various alternatives responsive to considers such as the location of the inlet, the outlet, controls, receivers for wireless communication and power and other functional aspects and as well as other modifications to operational characteristics depending on the implant location and orientation selected for the implanted kidney.

In still other embodiments, one or more of the design features described herein including without limitation those of one of the embodiments described in co-pending, commonly assigned U.S. Provisional Patent Application Ser. No. 62/xxx,xxx, filed Jul. 14, 2016, entitled “BIOCOMPATIBLE AND HEMOCOMPATIBLE MATERIAL AND FILTER,” (Atty Docket No. 14172-702.100) may be modified for use in or configured to provide advantages described herein into any of the components, systems, techniques and methods described in any of the following: WO2010088579A2; U.S. Pat. No. 7,540,963B2; US20090131858A1; WO2008086477A1; US20060213836A1; U.S. Pat. No. 7,048,856B2; US20040124147A1; US20120310136A1; WO2010088579A2; U.S. Pat. No. 7,540,963B2; US20090131858A1; U.S. Pat. No. 7,332,330B2; US20060213836A1; U.S. Pat. No. 7,048,856B2; US20040124147A1; WO2004024300A1; WO2003022125A2; US20030050622A1; WO2010057015A1; US20100112062A1; US20040167634A1; WO1998009582A1; U.S. Pat. No. 9,301,925B2; US20160002603A1; US20130344599A1; US20090202977A1; WO2007025233A1; US20120289881; US20130109088A1; U.S. Pat. No. 8,470,520B2; WO2013158283A1; U.S. Pat. No. 7,083,653; Nissenson A. R. a, Ronco C. b, Pergamit G. c, Edelstein M. c, Watts R. c; “The Human Nephron Filter: Toward a Continuously Functioning, Implantable Artificial Nephron System”; Blood Purif 2005; 23:269-274; (D01:10.1159/000085882); Jeremy J Song, Jacques P Guyette, Sarah E Gilpin, Gabriel Gonzalez, Joseph P Vacanti & Harald C Ott; “Regeneration and experimental orthotopic transplantation of a bioengineered kidney”, Nature Medicine, 19, 646-651; (2013); doi:10.1038/nm.3154; Madariaga M L, Ott H C., “Bioengineering kidneys for transplantation”, Semin Nephrol. 2014 July; 34(4):384-93. doi: 10.1016/j.semnephro1.2014.06.005. Epub 2014 Jun. 13; Song J J, Guyette J P, Gilpin S E, Gonzalez G, Vacanti J P, Ott H C., Regeneration and experimental orthotopic transplantation of a bioengineered kidney. Nat Med. 2013 May; 19(5):646-51. doi: 10.1038/nm.3154. Epub 2013 Apr. 14, William H. Fissell, IV, H. David Humes, Shuvo Roy, Aaron Fleischman. “Ultrafiltration membrane, device, bioartificial organ, and methods”; Patent: U.S. Pat. No. 7,540,963B2; Domenico Cianciavicchia, Claudio Ronco. “Wearble artificial kidney with regeneration system” Patent: EP2281591B1, each of which is incorporated herein by reference in its entirely for all purposes.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A material comprising:
a ceramic substrate having an outer surface(s) from which pores extend into said substrate; and
a coating over the surface layer(s) comprising a continuous layer of pyrolytic carbon which may infiltrate the substrate.

2. The material of claim 1, wherein the coating has a thickness of about 5 nm to 50 μm.

3. The material of claim 1, wherein the ceramic substrate is a ceramic tube filter.

4. The material of claim 3, wherein the tube filter comprises one or more channels.

5. The material of claim 1, wherein the ceramic substrate is a ceramic disk filter.

6. A material according to claim 1, wherein said substrate is formed of a ceramic material selected from the group consisting of the nitrides, carbides, or oxides of aluminum, silicon, boron, titanium, zirconium, or mixtures thereof.

7. The material of claim 1, wherein the cut off for filtering molecules is about 30 Da to 200,000 Da.

8. A material according to claim 1, wherein said coating provides greater biocompatibility and hemocompatibility than the unmodified ceramic substrate material.

9. A material according to claim 1 adapted and configured for use in a component or for integration within a housing or for positioning to filter human or animal blood as part of the improved operation of an implantable or external blood filtration system or a clinical or bedside blood filtration system.

10. A material according to claim 1, wherein the material is about 1 mm to 10 cm in width and 5 mm to 50 cm in length.

11. A method of manufacturing a filter, comprising
providing a tube filter comprising a ceramic substrate with an outer surface from which pores extend into the substrate;
mounting the tube filter between two mounting disks to form a mounted filter assembly;
placing the mounted filter assembly in a quartz reactor; and
pyrolizing a single layer of material comprising carbon on the ceramic substrate.

12. The method of claim 11, further comprising placing the quartz reactor in a tube furnace.

13. The method of claim 11 or 12, wherein the mounting disks comprise a disk comprising
an inner seat configured to seat an end of the ceramic tube filter; and
a plurality of holes configured to allow passage of gas.

14. The method of claim 11, wherein the inner seat comprises a hole through the disk.

15. The method of claim 11, wherein the pyrolizing occurs at temperatures between about 700° C. and 1200° C.

16. The method of claim 11, wherein at least 40% of the pores remain open during and after the pyrolizing.

17. The method of claim 11, wherein the pyrolytic coating layer is porous itself.

18. A hemofiltration device comprising
an outer housing;
an inlet port passing through the housing configured to receive a fluid;
an outlet port passing through the housing to remove flow from the device;
at least one ultrafiltration ceramic membrane inside the housing;
an arterial inlet chamber configured to join to a patient's artery and to the inlet port;
a venous outlet chamber configured to join to a patient's vein and to the outlet port; and
a cap on each end of the housing configured to seal the device and distribute flow of blood evenly to both ultrafiltration ceramic membranes.

19. The device of claim 18, wherein the housing comprises a biocompatible material.

20. The device of claim 18, wherein the housing comprises at least one of titanium, stainless steel, and PEEK.

21. The device of claim 18, wherein the patient's artery is the iliac artery

22. The device of claim 18, wherein the patient's vein is the iliac vein.

23. The device of claim 18, wherein at least one of the ultrafiltration ceramic membranes comprise tube filters.

24. The device of claim 18, wherein at least one of the ultrafiltration ceramic membranes comprises a tube filter.

25. The device of claim 18, wherein at least one of the ultrafiltration ceramic membranes comprises one or more channels.

26. The device of claim 25, further comprising biocompatible tubing connected to each channel.

27. The device of claim 18 wherein at least one of the arterial inlet chamber and the venous outlet chamber comprises a vascular graft.

28. The device of claim 18, wherein at least one of the caps comprises a barb.

29. The device of claim 18, comprising sealing plates positioned near the caps.

30. The device of claim 18, comprising a dialysis port configured for connection with a percutaneous port.

31. The device of claim 18, further comprising sealing O-rings at ends of the device.

32. The device of claim 18, wherein the membrane comprises a coating.

33. The device of claim 32, wherein the coating comprises at least one of a pyrolytic carbon and a diamond like carbon.

34. The device of claim 18, wherein the ceramic membrane comprises a diameter of about 25 mm.

35. The device of claim 18, wherein the ceramic membrane comprise a length of about 100 mm.

36. The device of claim 18, wherein the ceramic membranes comprise a pore size of about 30 Daltons to 200,000 Daltons.

37. The device of claim 18, wherein the filter comprise a filtration area of at least 0.1 m².

38. The device of claim 18, comprising a controller, valves and a pump on the outside of the patient connected to the device via a drive line.

39. The device of claim 18, wherein the ceramic membranes are configured to hold a volume of about 200 ml.

40. The device of claim 18, wherein the device is connected to renal artery and renal vein of a human kidney through dialysis port(s).

41. The device of claim 18, wherein the device is connected to renal artery and renal vein of an animal kidney through dialysis port(s).

42. The device of claim 18, wherein the device is connected to renal artery and renal vein of a human kidney through blood port (s).

43. The device of claim 18, wherein the device is connected to renal artery and renal vein of an animal kidney through blood port (s).

44. The device of claim 18, wherein the device is connected to another device through at least one of the blood port (s) or dialysis port (s) for further processing of the filtrate or blood.

45. The device of claim 18, wherein the device is connected to another device through at least one of the blood port (s) or dialysis port (s), where the combination of the devices can purify blood without any need to use dialysate.

46. The device of claim 18, wherein the device comprises two ultrafiltration ceramic membrane inside the housing.

47. The device of claim 18, wherein the filter is configured to concentrate uremic toxins in the filtrate and keep proteins such as albumin in blood.

48. A method for filtering blood, comprising
implanting a filtering device in a patient, the device comprising a housing; an inlet, an outlet, and two ultrafiltration ceramic membranes inside the housing;
connecting an inlet of the device to an artery of the patient; and
connecting an outlet of the device to a vein of the patient.

49. The method of claim 48, further comprising blood entering the device at about 1-2 psi.

50. The method of claim 48, further comprising pumping dialysate to the device.

51. The method of claim 50, wherein the dialysate is pumped at a pressure of about 0.5-15 psi.
